(19) United States
(12) Patent Application Publication
(10) Pub. No.: US 2026/0321413 A1
(43) Pub. Date: Oct. 15, 2026

(54) SYSTEM AND METHOD FOR EVALUATING A COGNITIVE AND PHYSIOLOGICAL STATUS OF A SUBJECT

(71) Applicant: COGME LTD., Haifa (IL)

(72) Inventors: Yogev HERZ, Kibbutz Ketura (IL); Steven Zvi LAPP, Ashdod (IL); Liat FEINBERG, Raanana (IL); Ilan BEN-OREN, Modiin (IL)

(21) Appl. No.: 19/101,403

(22) PCT Filed: Aug. 14, 2023
(86) PCT No.: PCT/IL2023/050849

§ 371 Date: Feb. 5, 2025

Related U.S. Application Data
(60) Provisional application No. 63/398,157, filed on Aug. 15, 2022.

(51) Int. Cl. G16H 40/67; A61B 5/00; A61B 5/16; A63B 24/00; G16H 50/30

(52) U.S. Cl. CPC G16H 40/67; A61B 5/0022; A61B 5/165; A61B 5/7264; A63B 24/0075; G16H 50/30

(57) ABSTRACT

Disclosed is a system for evaluating a cognitive and physiological status of a subject, including: a basic data module for inputting/uploading demographic and medical history data; a physiological sensor module including physiological sensors, a cognitive monitoring module including a personal assistance Application (PA App) operationally connected to a preprocessing unit and to an in-memory queue configured to temporarily store even logs, video/image data and audio data of the subject, recorded during questions, games/exercises presented to the subject, and to serve the data to the preprocessing unit for preprocessing; and a cloud-based server configured to receive the demographic and medical history data and; the signals obtained from the physiological sensor module and/or parameters/features; and the data preprocessed by the preprocessing unit; and to apply an AI algorithm on the received data to thereby compute a current and predicted cognitive and physiological status of the subject.

### TECHNOLOGICAL FIELD

The present disclosure generally relates to computer implemented method and system for holistic and personalized evaluation of a patient's cognitive and physiological status, in particular the system and method is suitable for prevention and early detection of delirium and its underlying cause/s.

### BACKGROUND

Delirium is a neuropsychiatric syndrome with a multifactorial etiology that occurs often in patients of Intensive Care Units (ICU). In fact, the prevalence rates of delirium in an ICU often exceeds 50%. Delirium is a serious change in mental abilities manifested by confused thinking and a lack of awareness of someone's surroundings. The disorder usually develops fast—typically within hours or a few days.

The etiology of delirium varies and can often be traced to numerous factors, including severe or long illness, an imbalance in the body, such as low sodium, medicine induced delirium, infection, surgery, or alcohol or drug use or withdrawal.

Symptoms of delirium are sometimes confused with symptoms of dementia. Delirium is associated with increased mortality, prolonged hospital stay, and long term effects such as decreased independent living, increased rate of institutionalization and increased risk to develop long-term cognitive impairment. Longer hospital stays and complications associated with delirium in the ICU lead to significantly higher costs of care. Accurate and early detection and treatment of delirium is the key to improving patient outcome and curbing delirium-related health care costs.

Currently, for the diagnosis of delirium in ICU patients a couple of validated screening questionnaires (such as CAM-ICU) are used. With these methods patients should be checked at least two to three times a day, but this is very difficult in most clinical settings. With the fluctuating character of delirium, delirious episodes are easily missed. Accurate and early detection methods may lead to more effective application of appropriate clinical interventions, leading to prevention of delirium or early detection thereof for better outcome and reduced mortality.

Hence, there is a need for a continuous and objective evaluation of patients' cognitive, physiological, and activity status to detect deterioration, evaluate the patient in an holistic manner and identify the underlying cause of delirium.

### SUMMARY

There is provided herein an AI-based platform for holistic and personalized evaluation of a patient's cognitive and physiological status.

Advantageously, the herein disclosed system and method enable assessment of a subject's risk of developing delirium, its early detection and preferably its underlying cause.

According to some embodiments, the system and method may further be configured to provide personalized recommendations for preventing and/or ameliorating and/or treating delirium in form of digital therapeutics platform. This digital therapeutic platform can be based on guidelines and recommendation of how to act upon identification of deterioration of the patient and/or identification of triggers, by the system AI, such as infection, metabolic imbalance, high or too low blood pressure, Acute respiratory distress syndrome, lack of sleep, sever patient unrest, pain, etc. Advantageously, the system and method may provide cognitive stimulations to maintain and enhance cognitive performances of patients and assist in orientation of place and time, thereby directly preventing delirium and/or cognitive or psychological decline, which can be considered a direct “digital therapeutic tool”. According to some embodiments, the hereindisclosed system and/or parts thereof (e.g. the PA App described hereinbelow) may be a digital therapeutic tool, prescribed to a patient, preferably, but not necessarily, under supervision and/or responsibility of medical staff.

Advantageously, the system and method employ a holistic approach which includes integrated monitoring of physiological and activity as well as cognitive parameters of patients, as well as changes therein. At times, changes in cognitive parameters may be indicative of a physiological deterioration. At other times, changes in physiological and activity parameters may be indicative of cognitive deterioration. At yet other times, a small but concurrent change in cognitive and physiological parameters may be indicative of physiological and/or cognitive deterioration. According to some embodiments, a correlation between cognitive changes and physiological changes and changes in activity may be utilized to detect an underlying cause of deterioration and assist caregivers in optimizing patient management and preferably preventing deterioration of the patient into delirium or other medical conditions. The AI system can be fed with additional data like changes in drugs to identify correlation of such changes with the status of the patient.

According to some embodiments, the herein disclosed system and methods and/or portions thereof, may be utilized for delirium screening and diagnostic tests, either alone or in conjunction with standard delirium tests, such as CAM and 4AT, thereby assisting in the “formal diagnosis” of delirium.

As a further advantage, the herein disclosed system and method may optimize patient management by providing data from the system to EMR databases and/or by incorporating data from EMRs into the system to detect changes in patient status and improve care. As a non-limiting example, new drug prescriptions can be evaluated, and recommendations provided so as to avoid certain classes of drugs for particular patients.

As a further advantage, the herein disclosed system and method may optimize patient management by providing a diagnostic aid tool, based on an AI expert system. The system may also bring up potential therapeutic suggestion based on AI expert system and guidelines or acceptable management algorithms. For example, the expert system can detect patient's dehydration by analyzing the difference in blood pressure in different postures, skin conductivity and/or blood work and recommend patient rehydration by oral intake or by saline infusion. Similarly, the system can detect specific abnormal cardiovascular condition and proposed a few options for treatment. As yet another example, the system may recommend a certain type of cognitive exercises to be conducted or to increase the frequency of cognitive training.

According to some embodiments, there is provided a system for evaluating a cognitive and physiological status of a subject, in particular to predict, identify and/or prevent delirium, the system comprising:
a basic data module comprising a user interface for inputting and/or uploading demographic data and medical history data of the subject;
a physiological sensor module comprising one or more physiological sensors,
a cognitive monitoring module comprising a personal assistance Application (PA App), the PA App comprising a preprocessing unit configured to present questions, games/exercises, and/or mental tracking exercises to the subject, and an in-memory queue configured to temporarily store even logs, video/image data and audio data of the subject, recorded during the questions, games/exercises, mental tracking exercises and to serve the stored data to the preprocessing unit for preprocessing; and
a server configured to receive:
the demographic data and the medical history data and/or on data derived therefrom;
signals obtained from the physiological sensor module and/or parameters/features derived therefrom; and
the data preprocessed by the preprocessing unit.

According to some embodiments, the server may be a cloud-based server. According to some embodiments, the server may be a server of a processing unit capable of conducting heavy-load processing including application of AI and big data analysis of large amount of data.

According to some embodiments, the server is configured to apply image analysis, machine learning, big-data analytics or natural language processing on the received data in order to derive additional physiological, cognitive and activity related parameters/features therefrom. Non-limiting examples of suitable such algorithms have been provided herein.

According to some embodiments, the server is configured to create/produce/generate a report visualizing the display the additional physiological, cognitive and activity related parameters/features and/or scores associated therewith. According to some embodiments create/produce/generating the report comprises organizing the additional physiological, cognitive and activity related parameters/features and/or scores associated therewith in a user friendly format. According to some embodiments, the server is configured to display the additional physiological, cognitive and activity related parameters/features and/or scores associated therewith on a display. According to some embodiments the displaying comprises organizing the additional physiological, cognitive and activity related parameters/features and/or scores associated therewith in a user friendly format.

According to some embodiments, the cloud-based unit is configured to apply an AI algorithm on the received data and/or on the additional physiological, cognitive and activity related parameters/features derived from the received data and/or on scores associated therewith to thereby compute a current and predicted cognitive and physiological status of the subject. It is thus understood that the received data and/or on the additional physiological, cognitive and activity related parameters/features derived from the received data and/or on scores associated therewith may serve as an input to an AI algorithm (also referred to herein as a “primary AI algorithm”) configured to integratively analyze all the input data and to predict and/or identify delirium (including pre-diagnosis delirium) based thereon.

According to some embodiments, the preprocessing unit may be configured for synchronous, real-time processing of the recorded audio by applying a voice recognition algorithm thereon, the voice recognition algorithm configured to recognize the voice of the subject and to cancel out environmental noise and other speakers' audio and preferably to identify a beginning and an end of a speech of the subject. According to some embodiments, the preprocessing unit may then time a next exercise, question or game based on the identified beginning and end of the subject's speech.

According to some embodiments, the preprocessing unit may be configured for synchronous, real-time processing of the event log, and to time a next exercise, question or game, based thereon.

According to some embodiments, the queue is configured to serve the recorded audio for asynchronous processing by the preprocessing unit, wherein the preprocessing comprises extracting voice features from the audio. and transmitting the voice features to the server.

According to some embodiments, the preprocessing unit is further configured to distort the recorded audio and subsequently transmit the distorted audio to the server for further (heavy load) processing. According to some embodiments, the preprocessing of the audio conducted by the preprocessing unit may be a minimal amount of processing, but sufficient to ensure the anonymity of the subject.

According to some embodiments, the further processing (conducted on the server) comprises extracting response content from the distorted audio, using one or more transcription algorithms and/or NLP or LLM models thereon.

According to some embodiments, the queue is configured to serve the stored image/video data for asynchronous preprocessing by the preprocessing unit, wherein the preprocessing comprises applying emotion extraction and key point extraction algorithms on the video/image data and transmitting the extracted emotions and key points to the server for further processing. According to some embodiments, the preprocessing of the image/video data conducted by the preprocessing unit may be a minimal amount of processing, but sufficient to ensure the anonymity of the subject.

According to some embodiments, the further processing (conducted by the server) comprises applying image analysis algorithms on the extracted emotions and key points.

According to some embodiments, the queue is configured to delete the stored data upon completion of preprocessing.

According to some embodiments, the questions, games/exercises, and/or mental tracking exercises are dynamic and/or personalized.

According to some embodiments, the preprocessing unit is configured to adjust a next question, game and/or exercise based on the preprocessed data.

According to some embodiments, the server is further configured to provide exercises/stimulations configured to prevent and/or ameliorate cognitive decline, based on the computed current and predicted cognitive and physiological status of the subject. According to some embodiments the PA App may thus serve as a digital therapeutic tool.

According to some embodiments, the server may be configured to determine a probable cause of a decline in the cognitive and/or physiological status of the subject, based on an integrated analysis of data obtained from the physiological sensor module and data obtained from the cognitive monitoring module. According to some embodiments, the server may be configured to determine a probable cause delirium of a patient, based on an integrated analysis of data obtained from the physiological sensor module and data obtained from the cognitive monitoring module.

According to some embodiments, the questions, games/exercises, and/or mental tracking exercises may be directed to evaluation of: general wellbeing of the subject, orientation of the subject, awareness of the subject, mental tracking, visual tracking and/or any combination thereof. According to some embodiments, the questions, games/exercises, and/or mental tracking exercises may be directed to improvement of: general wellbeing of the subject, orientation of the subject, awareness of the subject, mental tracking, visual tracking and/or any combination thereof.

According to some embodiments, the PA App communicates with the subject via a touch screen, via speaker/microphone, via a dedicated user interface or any combination thereof.

According to some embodiments, the server may be configured to rank the cognitive and physiological status of the subject with respect to a plurality of evaluated subjects.

According to some embodiments, the server may be configured to apply a classification algorithm on the demographic data and the medical history data, prior to the applying of the AI algorithm, thereby classifying the subject into a relevant patient group, representing the risk of the subject of developing delirium, and wherein the data derived from the demographic data and the medical history data comprises the risk of the subject to develop delirium.

According to some embodiments, the demographic data comprises gender, date of birth, country of birth, marital status, religion and/or any combination thereof.

According to some embodiments, the medical history data comprises admission type and date, transfers between departments, operations and procedures during hospitalization, admission and discharge dates, Charlson co-morbidity index (CCI), Norton scores, vital signs, medications used at home pre-hospitalization, medications during hospitalization, medications recommended at discharge, laboratory data, blood tests results, readmissions and reason for readmission, neurologic, geriatric and psychiatric consultations; and any combination thereof.

According to some embodiments, the one or more physiological sensors comprises a skin temperature sensor, a heart rate sensor, blood pressure sensor, an accelerometer, an oximeter, an ECG sensor, a respiration sensor, a sleep-tracker and/or any combination thereof.

According to some embodiments, the one or more parameters/features derived from the one or more physiological sensors comprises skin temperature, blood pressure changes during changes in posture, heart rate variability (HRV), HRV during rest, saturation, respiration rate, hours of sleep, depth of sleep and any combination thereof.

According to some embodiments the system may also function as a diagnosis of certain conditions including physiological conditions (such as a potential infection), cognitive/emotional conditions (as patient highly depressed), and lifestyle conditions (as patient did not sleep the last 2 nights). According to some embodiments, the physiological condition diagnosed may be the underlying cause of delirium.

According to some embodiments the system may further compute and display potential therapeutic recommendations, based on the diagnosed condition.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The invention will now be described in relation to certain examples and embodiments with reference to the following illustrative figures so that it may be more fully understood. It is stressed that the particulars shown are by way of example and for purposes of illustrative discussion of embodiments of the invention. In this regard, the description taken with the drawings makes apparent to those skilled in the art how embodiments of the invention may be practiced.

FIG. 1 schematically illustrates the herein disclosed system for holistic evaluation of a cognitive, physiological and activity related status of patients for early detection of delirium or other cognitive decline, according to some embodiments;

FIG. 2 shows exemplary questions and exercises that may be presented to patients via the herein disclosed PA App, according to some embodiments;

FIG. 3 schematically illustrates the operational setup of the herein disclosed cognitive monitoring module (PA App), according to some embodiments;

FIG. 4A shows an exemplary output of the herein disclosed physiological sensor module, according to some embodiments;

FIG. 4B shows an exemplary output of the herein disclosed cognitive monitoring module, according to some embodiments;

FIG. 5 is an illustrative example of key point extraction from image frames using asynchronous processing on a tablet installed with the herein disclosed PA App, according to some embodiments;

FIG. 6 shows an exemplary percentage of time with open eyes of an exemplary patient before and during delirium;

FIG. 7 illustrates an exemplary snapshot of abnormal cognitive, physiological and activity related parameters captured for a patient before diagnosis of delirium;

FIG. 8 illustrates an exemplary output of the herein disclosed holistic system over time along with delirium screening using 4AT and CAM;

FIG. 9 illustrates an exemplary output of the herein disclosed holistic system over time along with delirium screening using 4AT and CAM;

FIG. 10 illustrates an exemplary output of the herein disclosed holistic system over time along with delirium screening using 4AT and CAM;

FIG. 11 illustrates a normal sleep pattern and a sleep pattern obtained for a few patients suffering from delirium.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

For convenience, certain terms used in the specification, examples, and appended claims are collected here. Unless otherwise defined, all technical and scientific terms used herein have the same meaning as commonly understood by one of ordinary skills in the art to which this invention pertains.

According to some embodiments, disclosed herein is a system for evaluating a cognitive and physiological status of a subject.

As used herein, the terms “subject”, “patient” and “user” may be used interchangeably and may refer to any human individual undergoing hospitalization or other medical care, in particular individuals at risk of developing delirium or other cognitive impairment.

As used herein, the term “delirium” refers to a mental state of confusion, disorientation, and impaired ability to think or remember clearly. It usually starts suddenly, but as opposed to dementia and Alzheimer, it is often temporary and treatable. There are three types of delirium, namely hypoactive delirium, where the patient is not active and seems sleepy, tired, or depressed, hyperactive delirium, where the patient is restless or agitated and mixed delirium characterized by a shifting back and forth between hypoactive and hyperactive delirium. Due to this wide spectrum of behavior the diagnosis of delirium is complicated and often missed until the delirium is full-blown. Also, the etiology of delirium varies. Some of the more common causes include:
Alcohol or drugs, either from intoxication or withdrawal. This includes a serious type of alcohol withdrawal syndrome called delirium tremens. It usually happens to people who stop drinking after years of alcohol abuse.
Dehydration and electrolyte imbalances
Dementia
Hospitalization, especially in intensive care
Infections, such as urinary tract infections, pneumonia, and the flu
Medicines. This could be a side effect of a medicine, such as sedatives or opioids. Or it could be withdrawal after stopping a medicine.
Metabolic disorders
Organ failure, such as kidney or liver failure
Poisoning
Serious illnesses
Severe pain
Sleep deprivation
Surgeries, including reactions to anesthesia

According to some embodiments, the system includes:
a) a basic data module comprising a user interface for inputting and/or uploading demographic data and medical history data of the subject. According to some embodiments, at least some of the data may be retrieved from an Electronic Medical Record. to some embodiments, at least some of the data may be inputted via a dedicated user interface (e.g. at admission). According to some embodiments, data about the patients (inserted directly and/or from EMR) may be used to evaluate the initial risk (e.g. at intake) of an individual patient to develop cognitive decline during a stay in the hospital. In some embodiments the algorithm is also fed with data generated or collected during the hospital stay in order to allow dynamic adjustment of the risk.
b) a physiological sensor module comprising one or more physiological sensors. According to some embodiments, the physiological sensor may be a wearable sensor module, a bed sensor module or a combination thereof. The sensor can also be based on radar or camera sensors to evaluate physiological and activity parameters without patient contact. According to some embodiments, the data from the sensor module is collected and analyzed to detect changes in the physiological and/or activity (i.e., patient movement) condition of the patient, either as a stand-alone or in combination with other parameters, as further elaborated herein.
c) a cognitive monitoring module comprising a personal assistance Application (PA App), the PA App configured to present questions, games/exercises, and/or mental tracking exercises to a patient. The PA App is functionally associated with a processing unit (also referred to therein as “preprocessing unit”, of a local device such as, but not limited to the processer of a tablet or smartphone of the patient. The preprocessing unit comprises a in-memory queue configured to temporarily store captured data (event logs, video/image data and audio data of the subject recorded during use of the tablet, in particular during use of the PA App, and to serve the data to the preprocessing unit, without interfering with the patient's use of the PA App. According to some embodiments, the PA App is a dedicated software that operates on a tablet, cellphone, smart speakers such as Alexa, or the like. According to some embodiments, the PA App enables interaction with the patient, preferably a few times a day, to provide cognitive stimulation and to monitor cognitive and emotional changes. According to some embodiments, the monitoring is based on performance during activities prompted by the PA App, activities of choice performed by the user (such as games and web browsing) and image and voice analyses, as further elaborated herein. According to some embodiments, the PA App includes multiple guided questions with the aim of assessing different aspects of cognitive and physiological changes (e.g. utterances about feeling pain and the like) of that are typical to delirium and its potential underlying causes. Preferably, and in contrast to formal delirium screening tools such as the 4AT, the content of each session is unique, to prevent the patient from answering “automatically” and to prevent the patient from feeling under continuous examination.
The mode of communication with the PA App includes various options. Non-limiting examples include (each possibility and combination of possibilities is a separate embodiment):
   i. Q&A through touch screen.
   ii. Questions read through tablet speaker and/or presented on screen, answers provided vocally and/or with touch screen.
   iii. Voice only communication
   iv. Questions read through tablet speaker and/or screen, answers given through alternative human/machine interface as developed. This option may be particularly advantageous for patients suffering from illnesses such as ALS or at ICU.
   V. Video presented to the patient and questions follow that can be answered by touch or voice
d) a cloud-based server configured to receive:
   the demographic data and the medical history data and/or data derived therefrom; signals obtained from the physiological and/or activity sensor module and/or parameters/features derived therefrom; and
   the preprocessed video/image and audio data;
and;
to apply an AI algorithm on the received data to thereby compute a current and predicted cognitive and physiological status of the subject. According to some embodiments, the AI algorithm is configured to predict and identify early signs of cognitive and emotional changes and early stages of delirium in patients. According to some embodiments, the AI algorithm is configured to differentiate between delirium and other cognitive impairments such as early-stage dementia, dementia and Alzheimer. According to some embodiments, the cloud collection and analysis of raw and integrated data may be presented to the subject or to his/her caregiver at different levels of integration and interpretation and fusion, based on AI analysis, medical knowhow and combinations thereof.

Non-limiting examples of suitable AI-algorithms include convolutional neural network (CNN), recurrent neural network (RNN), long-short term memory (LSTM), auto-encoder (AE), generative adversarial network (GAN), Reinforcement-Learning (RL) and the like, as further detailed below. In other embodiments, the specific algorithms may be implemented using machine learning methods, such as support vector machine (SVM), decision tree (DT), random forest (RF), and the like. Each possibility and combination of possibilities is a separate embodiment. Both “supervised” and “unsupervised” methods may be implemented.

According to some embodiments, the preprocessing may be configured for synchronous and/or asynchronous processing of data.

As used herein the term “synchronous processing” refers to processing in which a request is sent and only upon completion of the task, work on the PA App can continue.

A used herein, the term “task” may refer to any manipulation of data requested from a processing unit to be performed. Non-limiting examples of optional tasks include: transcription of audio into text, emotion extraction, key point extraction, extraction of facial features from images etc. This process is often referred to as *blocking* (i.e. a next task cannot be conducted until a response is received). According to some embodiments, synchronous processing may be utilized to process data in real-time and with minimal delay.

According to some embodiments, the synchronous processing may be used for processing of task required to time a next step. As a non-limiting example, the synchronous processing may be applied on the recorded audio in order to identify a beginning and an end of a speech of the subject, wherein the beginning and end of the speech is utilized for timing a next exercise/question. In this instance, the synchronous processing comprises applying a voice recognition algorithm on the recorded data in order to identify the voice of the subject and distinguish it from other speakers' audio and environmental noise. Additionally or alternatively, the synchronous processing may be applied on the event log (e.g., number of clicks, time from last click and the like), in order to time a next exercise/question based thereon.

According to some embodiments, asynchronous processing (also referred to as non-blocking processing) refers to a processing in which an execution thread is not blocked after a request has been made, thereby allowing parallel execution of multiple requests. This is typically achieved by sending the requests to a in-memory queue configured to store data, to receive task requests to be executed on the data, and to serve the requests to the processing unit at a given time.

According to some embodiments, the queue may be used to store the data (event log, image/video and audio). According to some embodiments, the queue can be used to store data that need to be processed in real-time, such as data utilized for timing of a next step of an exercise. According to some embodiments, the queue can be used to store data for later analysis. According to some embodiments, the queue can be used for prioritization of task execution.

According to some embodiments, at least a portion of the preprocessing of the audio is asynchronous. That is, the audio may be stored in the queue and a task request served to the preprocessing unit. The task may for example include extraction of voice features (e.g., emotion extraction, intensity, pitch etc.) from the audio. Advantageously, the queue enables processing of sensitive material on a subject's local device (e.g., smartphone or tablet) without sending it to a server (thus ensuring privacy), while causing as little as possible interference to the smooth use of the local device as possible.

According to some embodiments, the emotion extraction may comprise applying a deep learning algorithm, such as but not limited to Torchaudio model on the audio. According to some embodiments, in addition to loading and saving audio data, Torchaudio provides a range of audio transformation options for manipulating and analyzing audio data including but not limited to: spectrogram (which provides a visual representation of the frequency content of an audio signal over time), resampling, feature extraction (extracting relevant features or characteristics from raw data that can be used as inputs to a machine learning model) and more.

According to some embodiments, the preprocessing is used for extraction of features which can only be obtained while working on the audio recording itself. However, the features extracted from the audio can be transmitted to a server (e.g., a cloud-based server) for further heavy-load processing, thereby minimizing the computational load on the preprocessing unit (such as a tablet processor), while preserving the privacy and/or anonymity of the subject.

According to some embodiments, upon extraction of the features that require the original audio, the preprocessing of the audio may further comprise distorting or otherwise anonymizing the audio. The distorted audio may then be transmitted to the server for further processing, such as, but not limited to content analysis of the audio (e.g. for determining correctness of answers in question that have a clear correct answer (Closed Questions), coherency of speech (for example reported by the patient as an answer to “Open Questions” such as question directed to: patient suffering from pain, patient did not sleep, patient does not have his glasses or hearing aid, etc.) without compromising the privacy and/or anonymity of the subject. Non-limiting examples of algorithms that may be applied on the distorted/manipulated audio comprises transcription algorithms, and NLP and LLM models and examples of such models include but are not limited to Open AI, Chat GPT version 3.5 and the open sourced Vicuna 13B and MPT-7B-Chat.

According to some embodiments, at least a portion of the preprocessing of the image/video data is asynchronous. That is, the image/video data may be stored in the queue and a task request served to the preprocessing unit for later processing. A non-limiting example of such task comprises applying emotion extraction and key point extraction algorithms on the stored video/image data.

Advantageously, by utilizing a queue the raw image/video data may be processed on the subject's local device (e.g., smartphone or tablet) without sending it to a server (thus ensuring privacy), while causing as little as possible interference to the smooth use of the local device as possible. According to some embodiments, multiple frames may be sent to the cloud and in the cloud machine and deep learning algorithms are used to detect and monitor changes associated with cognitive, emotional status of the patients, suffering from pain, etc. A non-limiting example of such analysis is the evaluation of eye opening during the session in the PA App.

According to some embodiments, once the features (e.g., emotion and key points) that can only be retrieved from the raw data have been extracted, the features can be transmitted to the server for further processing and the raw data filed be deleted. A non-limiting example of an algorithm suitable for emotion extraction comprises MobileNet Model. A non-limiting example of an algorithm suitable for key point extraction comprises MLKit Facemesh or MediaPipe.

According to some embodiments, the preprocessing is used for extraction of features which can only be obtained while working on the image/video recording itself. However, by transmitting the extracted features to a server (e.g., a cloud-based server) for further heavy-load processing, the computational load on the preprocessing unit (such as a tablet processor) is minimized, while the privacy and/or anonymity of the subject is maintained, since only extracted features, and not the recording itself, are transmitted.

According to some embodiments, the server-based processing comprises applying image analysis, machine learning and natural language (NLP) and LLMs algorithms on the data transmitted to the server. As a non-limiting example, image analysis algorithms may be applied on key points e.g. for tracking of eye movement, identification of head movement and the like.

Variables that can be inputted into the AI algorithm configured to determine the cognitive and physiological status of patients include, but are not limited to (each possibility and combination of possibilities is a separate embodiment):
1. Input variables for analysis:
   a. PA App variables:
      i. Number of session completed.
      ii. Number of correct answers.
      iii. Response time.
      iv. Video analysis of anonymized facial expressions such as closed eyes, smiles, signs of pain, etc.
      v. Sentiment voice analysis.
      vi. Performance in optional cognitive stimulation games and activities.
   b. Physiological sensor module variables:
      i. Raw measurements of vital signs and derivatives as HRV and deviations from baseline.
      ii. Steps and activity.
      iii. Sleep duration and quality.
      iv. Movements in bed
   c. Basic data variables (initial delirium risk):
      i. Demographics (age, gender, nationality)
      ii. Comorbidities
      iii. Presenting illness and procedures
      iv. Medications

According to some embodiments, at least some analyses will be performed automatically:
a. Upon admission:
   i. Calculation of overall delirium risk upon admission.
   ii. Assessment of variables that most significantly affect delirium for each patient.
b. Every hour:
   iii. incoming new relevant data (. e.g. “drop in cognitive performance”, “increase in body temperature”)
   iv. Processing of raw readings into a display-friendly format (e.g. raw sleep data into sum of hours slept).

According to some embodiments, the analyses may be used to display one or more of the following data:
a. Ranking of patients by delirium risk.
b. Stratification of patients into delirium risk bins (e.g. low, medium & high).
c. Important data points and trends indicating elevated delirium risk.
d. Incoming new measurements which may indicate elevated risk.
e. Summary of all raw data in the drill down screen of each patient and changes from baseline.

According to some embodiments, various image analysis, machine learning, natural language (NLP) and Large Language Models (LLMs) algorithms may be applied for the sever based analysis. Some of the algorithms may be used to extract additional parameters/features from the transmitted data, which additional parameters/features serve as an input to the AI model used to predict/identify patient deterioration and/or delirium (also referred to herein as the “primary AI model”).

As a non-limiting example, image analysis algorithms such as but not limited to Anisotropic diffusion, Hidden Markov models, Image editing, Image restoration, Independent component analysis, Linear filtering, Neural networks, Partial differential equations, Pixelation, Point feature matching, Principal components analysis, Wavelets or any combination thereof may be applied on the transmitted image data (e.g. key features) to extract additional information (e.g. eye opening time and in particular percent of eye's opening while the patient speaks vs. other periods of time), which additional feature (e.g. eye opening time) serves as an input to the primary AI model). Each possibility and combination of possibilities is a separate embodiment.

As another non-limiting example, an NLP and Large Language Models (LLMs) models such as, but not limited to Bidirectional Encoder Representations from Transformers (BERT), Robustly Optimized BERT Pretraining Approach (ROBERTa), GPT-3, ALBERT, XLNet, GPT2, StructBERT, Text-to-Text Transfer Transformer (T5), Efficiently Learning an Encoder that Classifies Token Replacements Accurately (ELECTRA), Decoding-enhanced BERT with disentangled attention (DeBERT) Dialog Flow, Spacy based models may be applied on the transmitted audio to extract emotions (e.g. the patient is sad, confused etc.), obtain information (e.g. the patient is suffering pain) and the like. Each possibility and combination of possibilities is a separate embodiment.

According to some embodiments, the primary AI model may be selected from convolutional neural network (CNN), recurrent neural network (RNN), long-short term memory (LSTM), auto-encoder (AE), generative adversarial network (GAN), Reinforcement-Learning (RL) and the like, as further detailed below. In other embodiments, the specific algorithms may be implemented using machine learning methods, such as support vector machine (SVM), decision tree (DT), random forest (RF), and the like. Each possibility and combination of possibilities is a separate embodiment. Both “supervised” and “unsupervised” methods may be implemented.

According to some embodiments, the queue is configured to automatically delete the image/video data therefrom upon transmittal of the extracted features (e.g., emotions and key points), thereby ensuring the anonymity of the subject and reducing the storage load of the local device.

According to some embodiment the questions, games/exercises, and/or mental tracking exercises are directed to evaluation of: general wellbeing of the subject, orientation of the subject, awareness of the subject, mental tracking, visual tracking and/or any combination thereof. Each possibility and combination of possibilities is a separate embodiment.

According to some embodiments, the questions, games/exercises, and/or mental tracking exercises are dynamic/personalized. That is the preprocessed data may be used as an input to an algorithm configured to change/adjust/pick next exercises, questions and/or games to be presented to the subject.

According to some embodiments, the PA App is further configured to provide exercises/stimulations configured to prevent and/or ameliorate delirium, based on the computed current and predicted cognitive and physiological status of the subject. According to some embodiments, the games/exercises may be designed to train cognition.

According to some embodiments, the server is further configured to determine a probable cause of a decline in the cognitive and/or physiological status of the subject, based on an integrated analysis of data obtained from the physiological sensor module and data obtained from the cognitive monitoring module and the AI model(s) applied thereon. According to some embodiments, the server is further configured to compute and output a treatment recommendation based on the probable cause of the identified cognitive status.

According to some embodiments, the PA App communicates with the subject via a touch screen, via speaker/microphone, via a dedicated user interface or any combination thereof. Each possibility and combination of possibilities is a separate embodiment.

According to some embodiments, the server is further configured to produce/output a report indicative of the subject's cognitive and physiological status. According to some embodiments, the report may be a written report. According to some embodiments, the report may be a virtual report. According to some embodiments, the report may be presented to the subject via the PA App. According to some embodiments, the report may be presented to a caregiver. For example, the report may be sent to a personal device (laptop, mobile device or the like) of the caregiver.

According to some embodiments, the server is configured to compute a score of the subject's cognitive and physiological status. According to some embodiments, the server is configured to rank each of a plurality of patients for example in an ICU unit, based upon which a prioritizing of the patients may be carried out.

According to some embodiments, the basic data unit includes an associated processor and/or dedicated software configured to apply an AI algorithm on the demographic data and the medical history data, thereby classifying the subject into a relevant patient group, the group indicative of the risk of the subject to develop delirium, and wherein the data derived from the demographic data and the medical history data comprises the risk of the subject to develop delirium. According to some embodiments, computing the risk that a patient will develop delirium (e.g., during a hospital stay) may be separate from the actual evaluation of the cognitive status of the patient. According to some embodiments, the computed risk may serve as an input to the comprehensive AI model configured to determine the cognitive and physiological state of the patient.

According to some embodiments, at least some of the demographic and/or medical history data may be pulled from an Electronic Medical Record (EMR). According to some embodiments, at least some of the demographic and/or medical history data may be inputted directly via a user interface. According to some embodiments, the user interface for inputting the demographic and/or medical history data may be accessible only to medical caregivers.

According to some embodiments, the demographic data inputted and/or uploaded to the basic data module includes one or more of gender, date of birth, country of birth, marital status, religion and/or any combination thereof. Each possibility and combination of possibilities is a separate embodiment.

According to some embodiments, the medical history data inputted and/or uploaded to the basic data module includes one or more of admission type and date, transfers between departments, operations and procedures during hospitalization, admission and discharge dates, Charlson co-morbidity index (CCI), Norton scores, vital signs, medications used at home pre-hospitalization, medications during hospitalization, medications recommended at discharge, laboratory data, blood tests results, readmissions and reason for readmission, neurologic, geriatric and psychiatric consultations; and any combination thereof. Each possibility and combination of possibilities is a separate embodiment.

According to some embodiments, the demographic and medical history data may be inputted to a trained machine learning algorithm operating at the cloud-based server, configured to compute and estimated initial risk of a patient to develop delirium. According to some embodiments, the initial risk may be the risk determined for the patient at intake/admission. According to some embodiments, the machine learning algorithm was trained on clinical data form 1,700 patients from the Rambam Health Care Campus (Haifa, Israel) which was split into training data and validation data. The training data was labelled with delirium diagnosis (or absence thereof) as obtained using the standard 4AT delirium screening test. The model was then trained to predict the 4AT score during the first week of hospitalization using only the data available at admission to the department. The model yielded an ROC-AUC of 0.76 for prediction of delirium (i.e. a 4AT score of 4 or more) during the first week of hospitalization.

According to some embodiments, the trained machine learning model is configured to stratify patients into segments from high to low risk (e.g. 3-5 segments). For example, on the validation data, the high-risk group contained 6% of patients who each had a 75% probability of developing delirium, while the low-risk group contained 50% of patients, each with only a 9% chance of developing delirium.

According to some embodiments, the machine learning algorithm can be “personalized” during the hospital stay by inputting additional data collected during the hospital stay, thereby improving and/or updating the prediction. According to some embodiments, the algorithm may be configured to provide a short-term prediction (i.e. for the next 48 hours).

According to some embodiments, the one or more physiological sensors comprises a skin temperature sensor, a heart rate sensor, blood pressure sensor, an accelerometer, an oximeter, an ECG sensor, a respiration sensor, a sleep-tracker and/or any combination thereof. Each possibility and combination of possibilities is a separate embodiment.

According to some embodiments, the one or more parameters/features derived from the one or more physiological sensors comprises skin temperature, blood pressure changes during changes in posture, heart rate variability (HRV), HRV during rest, saturation, respiration rate, hours of sleep, depth of sleep and any combination thereof. Each possibility and combination of possibilities is a separate embodiment. According to some embodiments, the sensor may be a non-contact sensor such as a camera or radar configured to monitor inter alia the activity of patients.

According to some embodiments, the one or more parameters/features derived from the one or more physiological sensors may be used to understand the underlying factors of an identified delirium. As a non-limiting example, skin temperature can be monitored to detect infection which often trigger delirium. As another non-limiting example, detection of changes in blood pressure when a patient changes his/her posture from standing to laying or sitting can be used for detection of dehydration, which is a known risk factor for delirium. This can be achieved by coupling blood pressure monitors along with a wearable that has an accelerometer. As yet another non-limiting example, Heart Rate Variability (HRV) data may be analyzed together with additional data, such as activity, to extract HRV during resting periods for detection of infection.

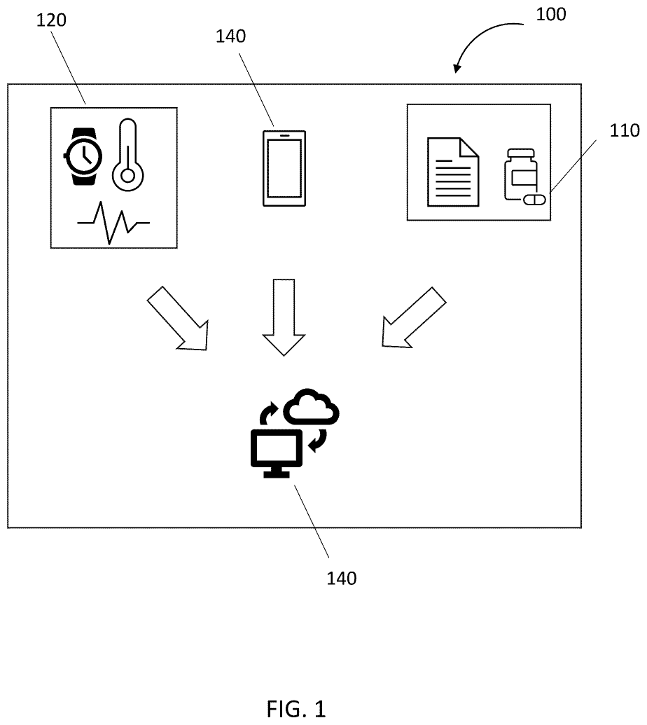

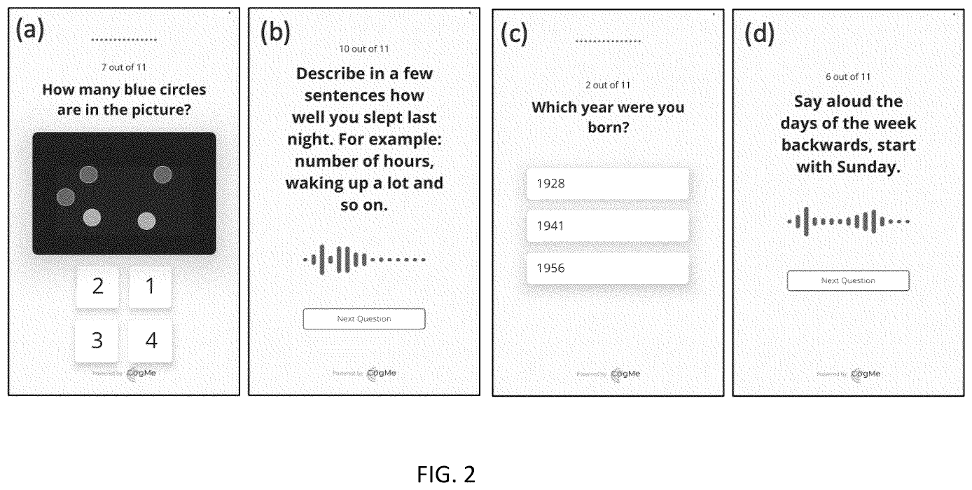

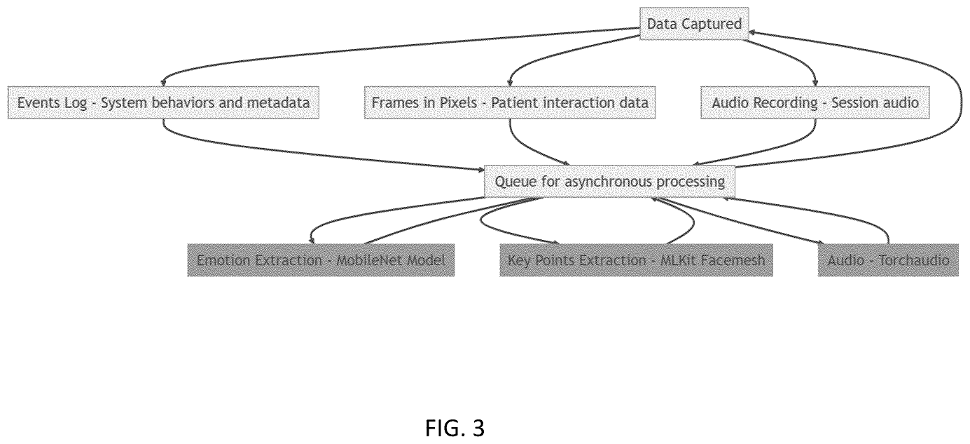

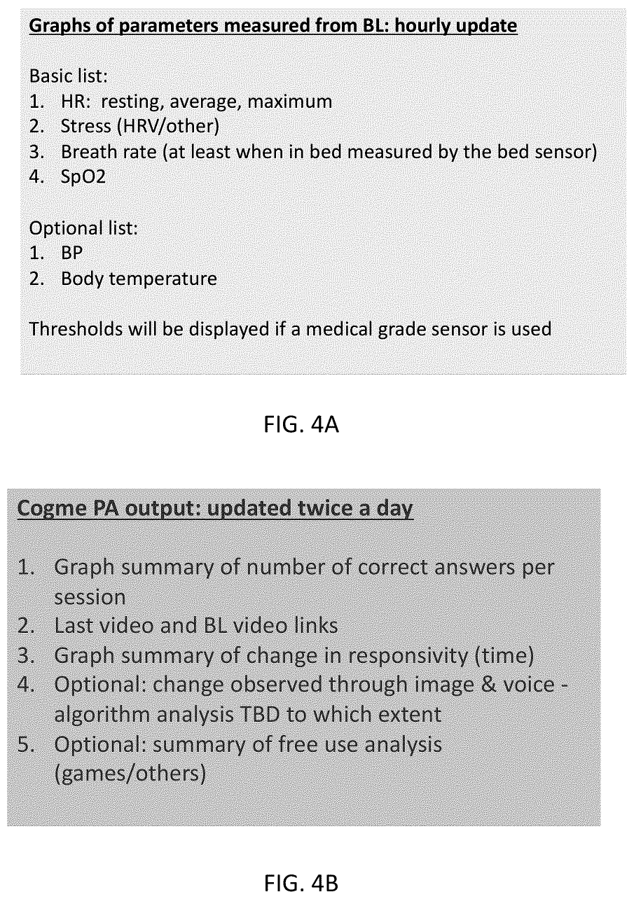

Reference is now made to FIG. 1, which schematically illustrates the herein disclosed system 100 for holistic evaluation of a cognitive, physiological and activity related status of patients for early detection of delirium or other cognitive decline. System 100 includes a basic data unit 110, configured to receive patients' medical history and demographic data. As elaborated herein, the data may according to some embodiments, be pulled from an EMR, inputted via a user interface (preferably accessible by medical personnel only). System 100 also includes a physiological sensor module 120, comprising one or more physiological sensors. According to some embodiments, the physiological sensors may be wearable sensors such as a wellness wearable (e.g., Apple or Garmin watch chest monitor), a bed sensor module (e.g. pulse oximeter, a capnograph, EMFIT or the like) or a combination thereof. System 100 also includes a cognitive monitoring module 130, preferably in the form of an application (also referred to herein as PA App) installed on a local device of the patients (e.g. a personal or dedicated mobile or tablet). Cognitive monitoring module 130 is configured to present questions, games/exercises, and/or mental tracking exercises to a patient, store event logs, audio text and image/video data, as well as to preprocess part of the data that cannot be sent to an external server without compromising the privacy/anonymity of the patients, as essentially elaborated herein. According to some embodiments, cognitive monitoring module 130 includes multiple questions, exercises and/or games to which the patients may respond/interact with, preferably via voice and/or touch communication. Suitable questions and exercises are illustrated in FIG. 2 and are all aimed at assessing different cognitive factors known to be associated with delirium. Preferably, and in contrast to formal delirium screening tools, such as 4AT, the content of each session presented via cognitive monitoring module 130 is unique, thereby preventing patients from answering “automatically” or from feeling under continuous examination. Further details of cognitive monitoring module 130 and its operational setup are illustrated with respect to FIG. 3. The output of each of basic data unit 110, physiological sensor module 120 and cognitive monitoring module 130 are all transmitted to an external server 140 for further individual processing. Ultimately all the processed data is inputted into an AI algorithm configured to predict an upcoming change in the subjects cognitive and/or physiological status and/or for identifying early stages of cognitive and/or physiological decline (specifically predicting and/or identifying early-stage delirium (preferably prior to manifestation of symptoms immediately identifiable by caregivers and/or family), as further elaborated herein. For each patient, external server 140 may be configured to provide an output (e.g., via display on a display) the data obtained from the physiological sensor module 120, preferably along with trends of changes from baseline (as for example as illustratively shown in FIG. 4A), cognitive performance data obtained from cognitive monitoring module 130 (as for example as illustratively shown in FIG. 4B) and/or the determined cognitive and physiological status of the patient. Each possibility and combination of possibilities is a separate embodiment. In addition to the evaluation of each individual patient, external server 140 may further to generate a summarizing report of all patients connected to system 100, thereby enabling ranking the patients according to their risk of developing delirium, preferably along with selected real-time readings from physiological sensor module 120 and cognitive monitoring module 130.

Reference is now made to FIG. 2 which illustrates non-limiting examples of suitable questions that may be used for the PA App:
(a) Graphical Exercises (some simple and some more complex).
(b) Open Questions: For example, the patient may be asked an open question (with and without option to answer through screen). Such as “How do you feel? ”, “Do you feel any pain?”, “How did you sleep?”. These questions provide insight into the patient's capability of communicating. And the answers are analyzed for content since issues of pain can be a significant contributor to delirium. Communication with the patient, specifically around open questions enable analyses of additional attributes such as facial expressions, voice etc. regardless of content, but rather in terms of the capability of talking fluently, tone of voice, emotions all which are often also affected by delirium. Importantly, the open question, while requiring more heavy computational processing, were found to be of uttermost importance, since some patients (e.g., patients suffering from dementia) find the open questions much easier to deal with (they may for example just require the patient to freely answer in speech without requiring clicking, touching dedicated parts of screen, or otherwise interacting directly with the PA App).
(c) Orientation Questions: For example, the patient may be asked a question indicating whether he or she is aware of his/her current location, current time or some of their biographic details. Such questions are indicative of delirium and appear in common mental tests for the elderly, such as AMT4 and the 4AT.
(d) Mental tracking: A non-limiting example of mental tracking includes a request to say aloud a complex series of numbers/words, such as the days of the week backward, months of the year backwards or to count in steps of 3. The test is analyzed for success rate and response time. Such tests have been shown to be indicative of attention and cognitive functions.

Reference is now made to FIG. 4, which schematically illustrates the data flow within the PA App.
Data Capturing: This is the initial stage where data is captured from the PA App. This includes event logs, frames in pixels, and audio recordings.
Events Log: is where all system-level behaviors, such as button clicks and screen displays, are logged. The metadata for each event, such as the time it occurred, is also captured. This data is stored, for example in a JSON file, for each unique session.

1Frames in Pixels: represents the visual data of the patient interacting with the tablet during a session. This data is enqueued for asynchronous processing, which includes deploying face keypoint facemesh models and emotion extraction models.
Audio Recording: The audio of the entire session is recorded for further analysis at the local device as well as in the cloud.
Queue for Asynchronous Processing: This is where the data from the Events Log, Frames in Pixels, and Audio Recording is enqueued for processing. The processed data can then be used for various purposes, such as emotion extraction. The queue may for example be implemented in java source code. The queue may also support a publish-subscriber pattern, allowing for dynamic customization of sessions, based on the patient's needs. For example, if the emotion extraction model detects that the patient is sad, it could push data back into the queue to adjust the difficulty level of a next PA session.
Emotion Extraction: This is where the emotion extraction model processes the data in the queue. Here the model used is the MobileNet model from the Python repository at HSE University, however it is understood that other models may likewise be applicable and as such are within the scope of this disclosure. The model predictions are stored with timestamp and values of the scores for one or more of the following emotions: Anger, Disgust, Fear, Happiness, Neutral, Sadness, Surprise.
Key Points Extraction: This is where the key points from the patient's face are extracted. Here the model used is the MLKit Facemesh, however it is understood that other models may likewise be applicable and as such are within the scope of this disclosure.
Audio: This is where the audio data is processed. Here the model used is Torchaudio, however it is understood that other models may likewise be applicable and as such are within the scope of this disclosure. For example, in addition to the emotion extraction based on Image analysis, the open-source py AudioAnalysis can be used for emotion extraction in particular.

It is understood that the strategy is to perform minimum required processing on the local device (e.g. tablet or mobile phone) to ensure smooth interaction with the patient, while at the same time minimizing transfer of sensitive patient video, image and/or audio information. Heavy computation is preferably done in the cloud, as further elaborated herein. Any calculation related to raw data or which is relevant to the session itself (e.g. timing of next question or exercise) may be done on the local device, when required online during the session.

In order to reduce noise in the data, while maximizing sensitivity to detect changes in patient condition, the system may extract same parameters using various (2 or more) different modalities. This, however, requires extensive computing power and can be done in the cloud only. For example, emotional and cognitive changes can be evaluated from image analysis (by MobileNet), audio tone analysis (by pyAudioAnalysis) with and from evaluation of the content of the audio answered and analyzed with LLM model (by Vicuna). The AI based expert system can analyze the features from the different modalities and decide which to use for further analysis and/or which to present to staff. Similarly, the AI system in the cloud can use different features extracted from one or more of the various modalities and fuse them (using data fusion techniques) to present a fused/integrated score, preferably fused scores of for different aspects, like cognitive, emotional, physiological, activity level, etc.

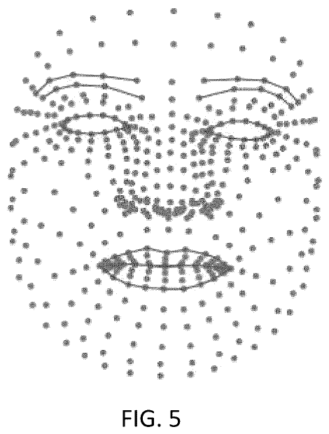

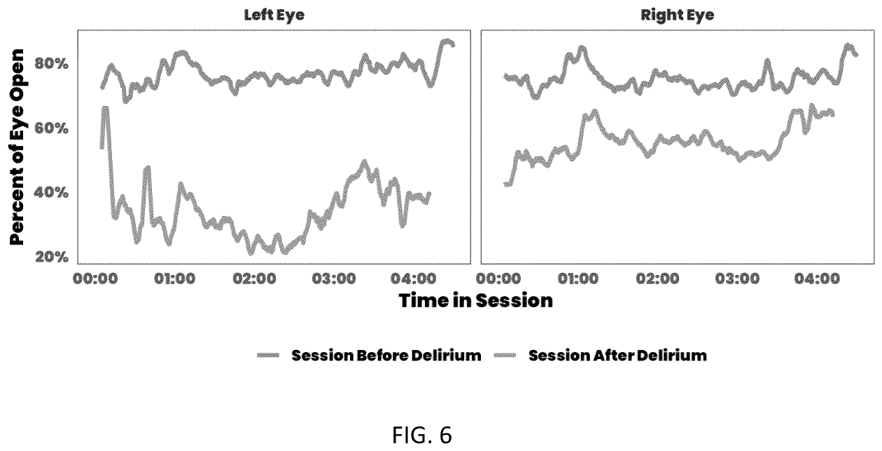

Reference is now made to FIG. 5, which is an illustrative example asynchronous analysis, conducted on a tablet installed with the herein disclosed PA App, to extract the key points for face analysis. Here a single frame is shown, however during use of the PA App, multiple frames are sent to the cloud and in the cloud machine and deep learning algorithms are used to detect and monitor changes associated with the cognitive, emotional and physiological status of the patients A non-limiting example of an analysis that may be conducted at the server is the evaluation of eye opening during the PA App session. As seen from FIG. 6, during delirium patients tend to maintain their eyes closed for extended periods of time, thus indicating its importance as a parameter for evaluation of delirium.

Although some embodiments of the invention are not limited in this regard, discussions utilizing terms such as, for example, “processing,” “computing.” “calculating,” “determining,” “establishing”. “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

As used herein, the terms “approximately”, “essentially” and “about” in reference to a number are generally taken to include numbers that fall within a range of 5% or in the range of 1% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

As used herein, the singular forms “a,” “an” and “the” include plural referents unless the context clearly dictates otherwise.

As used herein, “optional” or “optionally” means that the subsequently described event or circumstance does or does not occur, and that the description includes instances where said event or circumstance occurs and instances where it does not.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods, according to some embodiments, may be described in a specific sequence, the methods of the disclosure may include some or all of the described stages carried out in a different order. In particular, it is to be understood that the order of stages and sub-stages of any of the described methods may be reordered unless the context clearly dictates otherwise, for example, when a latter stage requires as input an output of a former stage or when a latter stage requires a product of a former stage. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications, and variations that are apparent to those skilled in the art may exist. Accordingly, the disclosure embraces all such alternatives, modifications, and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Example 1—Clinical Trial of the Herein Disclosed Holistic System for Delirium Evaluation

A clinical trial of the herein disclosed holistic system for detecting delirium before diagnosis was conducted at the Rambam Hospital, Israelx

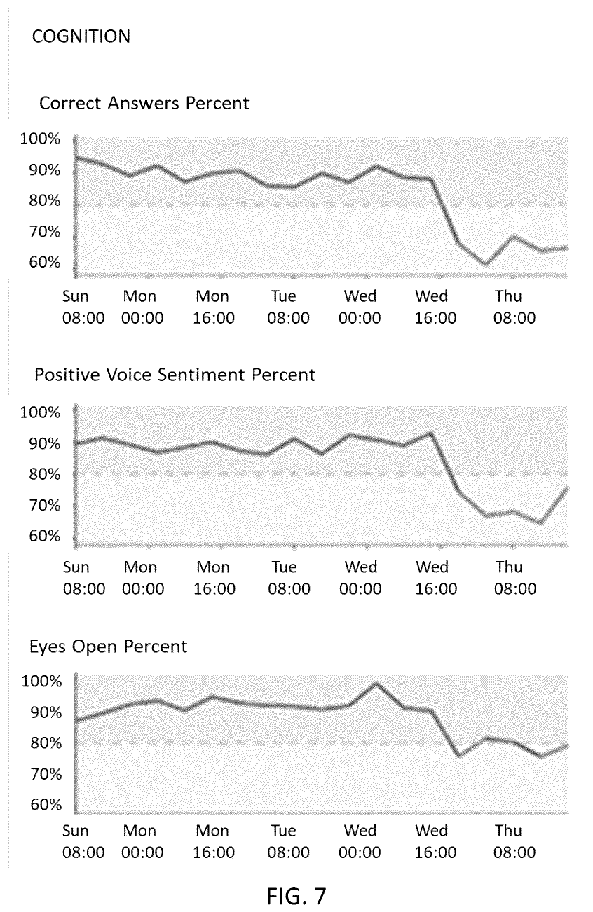

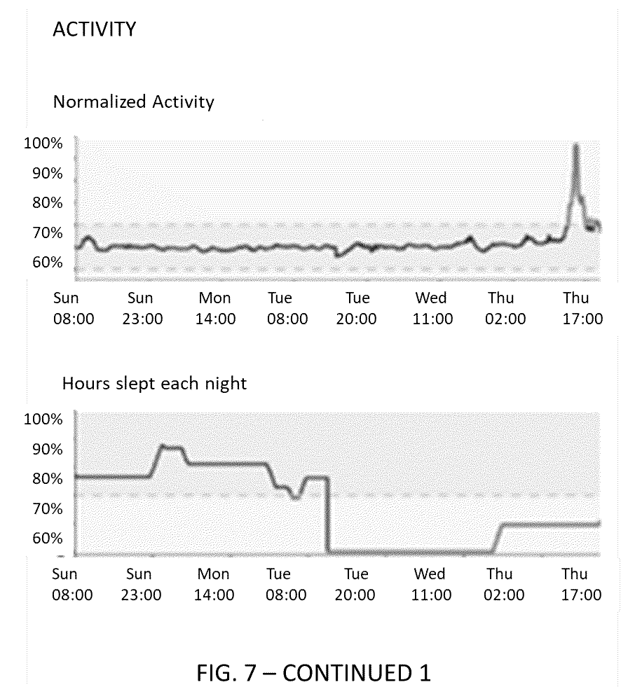

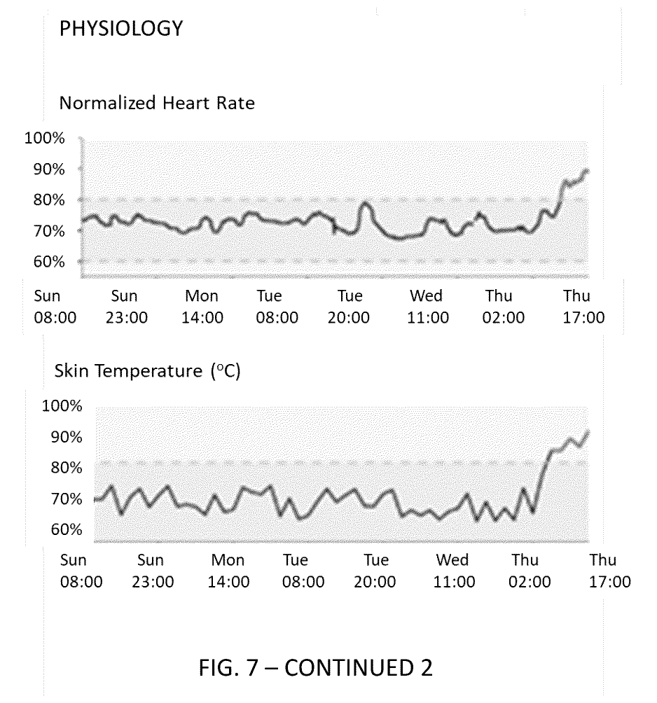

FIG. 7 shows a snapshot of abnormal parameters that captured for a specific patient at a certain time. As can be seen in this example, several cognitive, physiological and activity related parameters that deviate from base line and from the typical range (light blue band) were identified.

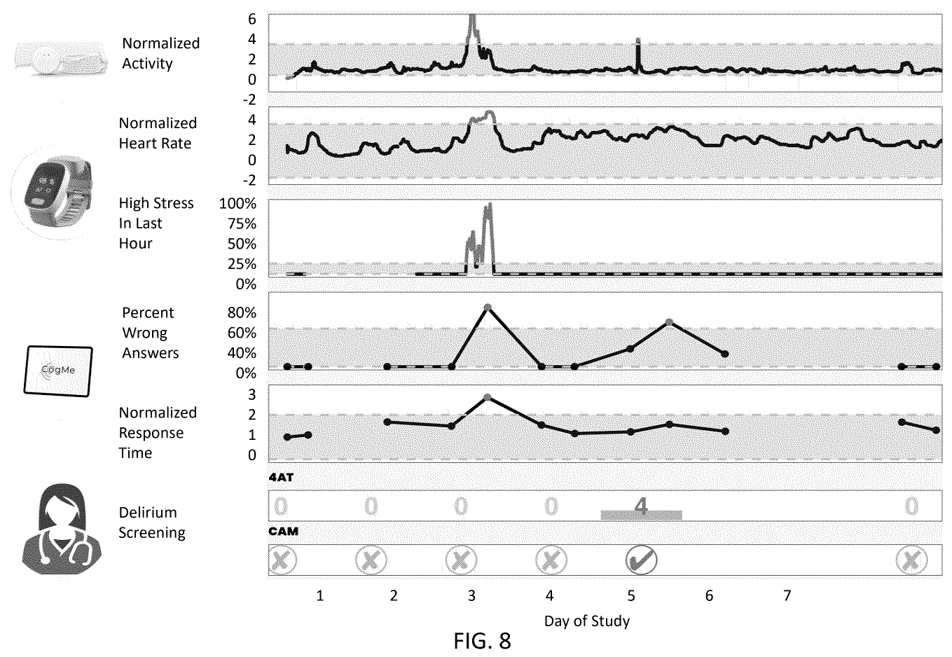

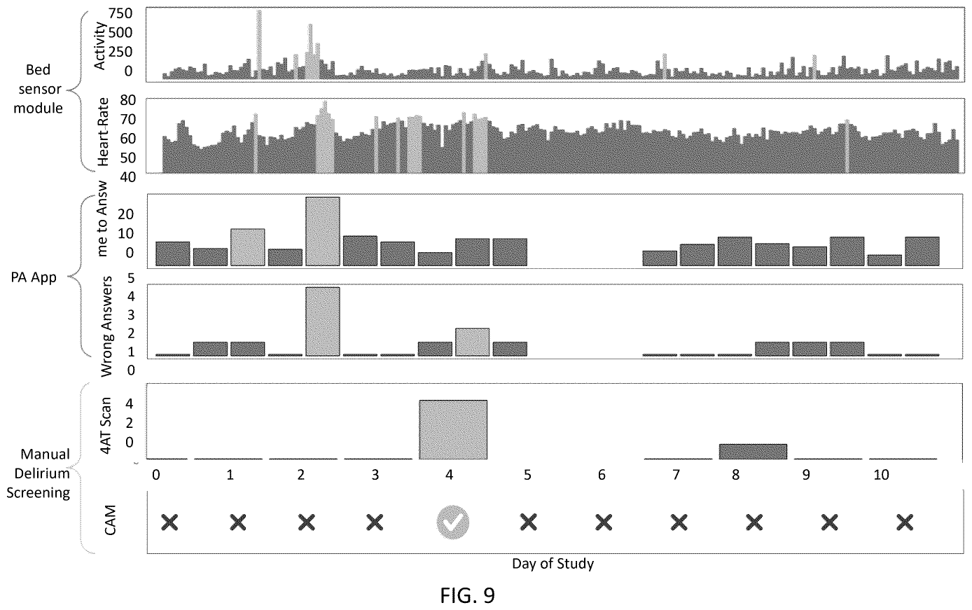

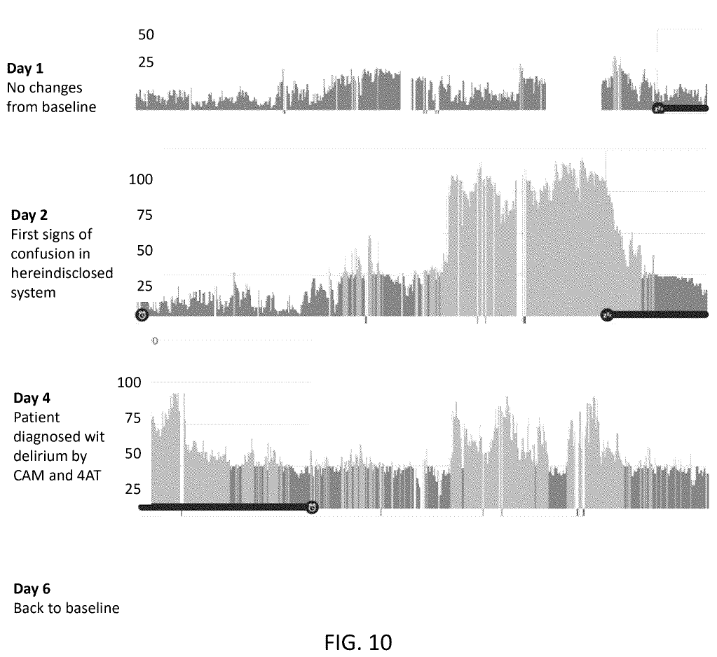

FIG. 8-FIG. 10, show additional illustrative examples of outputs obtained from the hereindisclosed holistic system for pre-diagnose detection of delirium in another patient. As seen from the figures, a formal diagnosis of delirium using delirium screening (4AT) and diagnosis with CAM was provided on day 5 of the study (days 6 and 7 were weekend days, and no delirium screening was conducted). However, the hereindisclosed system identified various abnormalities in physiological and cognitive parameters 2 days prior. It is understood to those skill in the art that such early indication may be paramount for initiating early treatment which in turn may result in reduced severity of the delirium and potentially preventing it all together. As further seen from the figures evaluating only physiological parameters, cognitive parameters or activity related parameters may result in false positives and false negatives, while an integrated analysis clearly enhances the reliability of the results. It is also worth noting that the continuous monitoring and analysis by the herein disclosed holistic system provided new insights that were not previously considered to be relevant or of valuable in the field. For example, a known study looking at “Heart rate variability in intensive care unit patients with delirium” concluded: “No difference could be found between patients with (N=13) and without (N=12) delirium by comparing the mean (=standard deviation) of the HFnu (75±7 versus 68±23) and the LF: HF ratio (−0.7±1.0 versus−0.1±1.1) [Irene J Zaal, et al, J Neuropsychiatry Clin Neurosc. 2015; 27(2): e112-6]. The study thus concludes that autonomic function is not altered in ICU delirium.”, while in fact, as is shown in FIG. 10 herein, the intraday variability of HRV brings valuable clinical information that is concealed when studying averages. Moreover, the value of monitoring intraday analysis of HRV, while being informative in itself, was bolstered when evaluated in the context of other parameters/features of the patients enabled reliable early, pre-diagnosis detection of delirium thereby facilitating early preventive measures being taken.

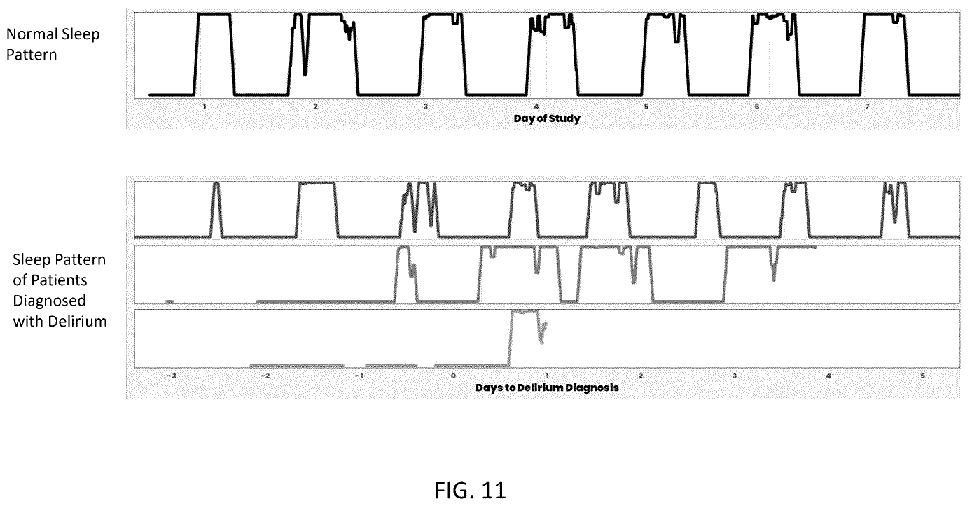

FIG. 11 specifically illustrates the sleep pattern of the patient before and during delirium.

### Example 2—Retrospective Study for Computing Initial Delirium Risk

1,700 patients hospitalized at the Rambam Hospital, Israel were labeled for presence or absence of delirium as diagnosed using the 4AT screening test.

A large plurality of medical history parameters and demographic data served as input values. Data of about half of the patients was used for training while the other half was used for validation. An ROC-AUC of 0.76 for prediction of delirium onset during the first week of admission was obtained, indicating the ability of the model to group patients based on their risk of developing delirium during the first week of hospital stay.

The model is currently being enhanced by adding dataset of 35,000 patients and 200,000 admissions.

The is further being expanded to a dynamic model in which data accumulated throughout hospitalization is used for updating of the prediction.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1-26. (canceled)

27. A system for evaluating a cognitive and physiological status of a subject, the system comprising:
a basic data module comprising a user interface for inputting and/or uploading demographic data and medical history data of the subject;
a physiological sensor module comprising one or more physiological sensors,
a cognitive monitoring module comprising a personal assistance Application (PA App), the PA App comprising a preprocessing unit configured to present questions, games/exercises, and/or mental tracking exercises to the subject, and an in-memory queue configured to temporarily store even logs, video/image data and audio data of the subject, recorded during the questions, games/exercises, mental tracking exercises and to serve the stored data to the preprocessing unit for preprocessing; and
a cloud-based server configured to receive:
i. the demographic data and the medical history data and/or on data derived therefrom;
ii. signals obtained from the physiological sensor module and/or parameters/features derived therefrom; and
iii. the data preprocessed by the preprocessing unit;
and to apply an AI algorithm on the received data to thereby compute a current and predicted cognitive and physiological status of the subject.

28. The system of claim 27, wherein the preprocessing unit is configured for synchronous, real-time processing of the recorded audio by applying a voice recognition algorithm thereon, the voice recognition algorithm configured to recognize the voice of the subject and to cancel out environmental noise and other speakers' audio and to identify a beginning and an end of a speech of the subject.

29. The system of claim 28, wherein the preprocessing unit is configured to time a next exercise, question or game based on the identified beginning and end of the subject's speech.

30. The system of claim 27, wherein the preprocessing unit is configured for synchronous, real-time processing of the event log, and to time a next exercise, question or game, based thereon.

31. The system of claim 27, wherein the queue is configured to serve the recorded audio for asynchronous processing by the preprocessing unit, wherein the preprocessing comprises extracting voice features from the audio. and transmitting the voice features to the server.

32. The system of claim 31, wherein the preprocessing unit is further configured to distort the recorded audio and subsequently transmit the distorted audio to the server for further processing, thereby ensuring the anonymity of the subject.

33. The system of claim 32, wherein the further processing comprises extracting response content from the distorted audio, using one or more transcription algorithms and/or NLP/LLM models thereon.

34. The system of claim 27, wherein the queue is configured to serve the stored image/video data for asynchronous preprocessing by the preprocessing unit, wherein the preprocessing comprises applying emotion extraction and key point extraction algorithms on the video/image data and transmitting the extracted emotions and key points to the server for further processing.

35. The system of claim 34, wherein the further processing comprises applying image analysis algorithms on the extracted emotions and key points.

36. The system of claim 27, wherein the queue is configured to delete the stored data upon completion of preprocessing.

37. The system of claim 27, wherein the questions, games/exercises, and/or mental tracking exercises are dynamic and/or personalized and wherein the preprocessing unit is configured to adjust a next question, game and/or exercise based on the preprocessed data.

38. The system of claim 27, wherein the server is further configured to provide exercises/stimulations configured to prevent and/or ameliorate cognitive decline, based on the computed current and predicted cognitive and physiological status of the subject.

39. The system of claim 27, wherein the server is further configured to determine a probable cause of a decline in the cognitive and/or physiological status of the subject, based on an integrated analysis of data obtained from the physiological sensor module and data obtained from the cognitive monitoring module.

40. The system of claim 27, wherein the PA App communicates with the subject via a touch screen, via speaker/microphone, via a dedicated user interface or any combination thereof.

41. The system of claim 27, wherein the server is further configured to rank the cognitive and physiological status of the subject with respect to a plurality of evaluated subjects.

42. The system of claim 27, wherein the server is further configured to apply a classification algorithm on the demographic data and the medical history data prior to the applying of the AI algorithm, thereby classifying the subject into a relevant patient group, representing the risk of the subject of developing delirium, and wherein the data derived from the demographic data and the medical history data comprises the risk of the subject to develop delirium.

43. The system of claim 27, wherein the one or more physiological sensors comprises a skin temperature sensor, a heart rate sensor, blood pressure sensor, an accelerometer, an oximeter, an ECG sensor, a respiration sensor, a sleep-tracker and/or any combination thereof and wherein the one or more parameters/features derived from the one or more physiological sensors comprises skin temperature, blood pressure changes during changes in posture, heart rate variability (HRV), HRV during rest, saturation, respiration rate, hours of sleep, depth of sleep and any combination thereof.

44. The system of claim 27, wherein the processing unit is further configured to output a suggested diagnosis of conditions associated with delirium, wherein the associated conditions comprise physiological conditions, cognitive/emotional conditions, and lifestyle conditions.

45. The system of claim 24, wherein the processing unit is further configured to produce a report indicative of the subjects cognitive and physiological status and to output a potential therapeutic recommendation.

46. A computer implemented method for evaluating a cognitive and physiological status of a subject, the method comprising:
obtaining via input and/or uploading demographic data and medical history data of the subject;
receiving a plurality of signals from a physiological sensor module comprising one or more physiological sensors,
receiving data from a cognitive monitoring module comprising a personal assistance Application (PA App), the PA App configured to present questions, games/exercises, and/or mental tracking exercises to the subject, to store in an in-memory queue even logs, video/image data and audio data of the subject, recorded during the questions, games/exercises, mental tracking exercises and to serve the stored data for preprocessing;
transmitting to a cloud-based server:
iv. the demographic data and the medical history data and/or on data derived therefrom;
v. signals obtained from the physiological sensor module and/or parameters/features derived therefrom; and
vi. the data preprocessed by the preprocessing unit; and
and at the cloud based server, applying an AI algorithm on the received data to thereby compute a current and predicted cognitive and physiological status of the subject.
